(19) United States
(12) Patent Application Publication
(10) Pub. No.: US 2007/0032668 A1
(43) Pub. Date: Feb. 8, 2007

(54) Semi-synthesis of taxane intermediates from a mixture of taxanes

(76) Inventors: Ragina Naidu, Burnaby (CA); Samuel Siang Kiang Foo, Vancouver (CA)

Correspondence Address: SEED INTELLECTUAL PROPERTY LAW GROUP PLLC, 701 FIFTH AVE, SUITE 5400, SEATTLE, WA, 98104, US

(21) Appl. No.: 11/452,103

(22) Filed: Jun. 12, 2006

Related U.S. Application Data
(63) Continuation of application No. 10/831,648, filed on Apr. 23, 2004, now abandoned.

(51) Int. Cl. A61K 31/337; C07D 305/12

(52) U.S. Cl. 549/510

(57) ABSTRACT

A process is provided for the semi-synthesis of taxane intermediates useful in the preparation of paclitaxel and docetaxel, in particular, the semi-synthesis of 10-deacetylbaccatin III and baccatin III, and derivatives thereof, from a mixture of taxanes.

### CROSS-REFERENCE TO RELATED APPLICATION

This application is a continuation of U.S. patent application Ser. No. 10/831,648, filed Apr. 23, 2004, now pending, which application is incorporated herein by reference in its entirety.

### BACKGROUND OF THE INVENTION

1. Field of the Invention

The present invention relates to the semi-synthesis of taxane intermediates useful in the preparation of paclitaxel and docetaxel, in particular, the semi-synthesis of 10-deacetylbaccatin III and baccatin III, and derivatives thereof, from a mixture of taxanes.

2. Description of the Related Art

The taxane family of terpenes has received much attention in the scientific and medical community because members of this family have demonstrated broad spectrum anti-leukemic and tumor-inhibitory activity. A well-known member of this family is paclitaxel (1, Taxol).

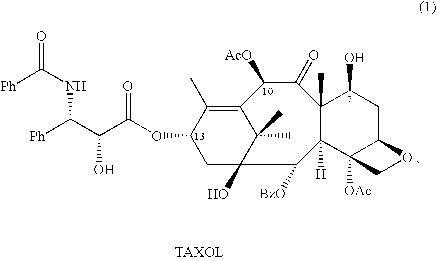

Paclitaxel was first isolated from the bark of the pacific yew tree (Taxus brevifolia) in 1971, and has proved to be a potent natural anticancer agent. For example, paclitaxel has been found to have activity against different forms of leukemia and against solid tumors in the breast, ovary, brain, and lung in humans.

This activity has stimulated an intense research effort over recent years, including the search for other taxanes having similar or improved properties, and the development of synthetic pathways for making taxanes such as paclitaxel. One result from this research effort was the discovery of a synthetic analog of paclitaxel, docetaxel (2, more commonly known as taxotere). As disclosed in U.S. Pat. No. 4,814,470, taxotere has been found to have very good anti-tumor activity and better bio-availability than paclitaxel. Taxotere is similar in structure to paclitaxel, having t-butoxycarbonyl instead of benzoyl on the amino group at the 3′ position, and a hydroxyl group instead of the acetoxy group at the C-10 position.

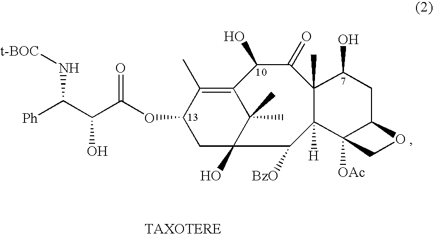

Taxanes are structurally complicated molecules, and the development of commercially viable synthetic methods to make taxanes has been a challenge. A number of semi-synthetic pathways have been developed, which typically begin with the isolation and purification of a naturally occurring material and then its conversion to the taxane of interest. For example, paclitaxel and taxotere may be prepared semi-synthetically from 10-deacetylbaccatin III or baccatin III as set forth in U.S. Pat. No. 4,924,011 to Denis et al. and U.S. Pat. No. 4,924,012 to Colin et al. or by the reaction of a β-lactam and a suitably protected 10-deacetylbaccatin III or baccatin III derivative as set forth in U.S. Pat. No. 5,175,315 to Holton et al. or U.S. patent application Ser. No. 10/683,865, which application is assigned to the assignee of the present invention. 10-deacetylbaccatin III (10-DAB, 3) and baccatin III (BACC III, 4) can be separated from mixtures extracted from natural sources such as the needles, stems, bark or heartwood of numerous Taxus species and have the following structures.

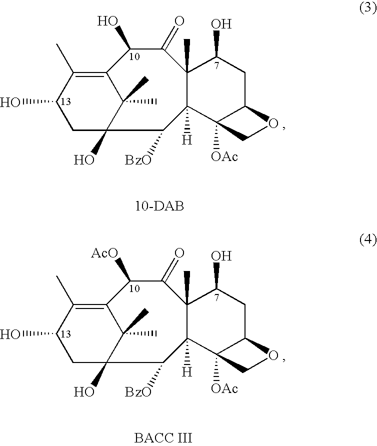

Although, much of the research towards the semi-synthesis of paclitaxel and taxotere has involved 10-deacetylbaccatin III as the starting material, other taxanes from the Taxus species, such as 9-dihydro-13-acetylbaccatin III (9-DHB, 5), present in the Canadian yew (Taxus Canadensis), cephalomannine (6), 10-deacetyl taxol (10-DAT, 7), 7-xylosyl taxol (8) and 10-deacetyl-7-xylosyl taxol (9) have been collected and identified.

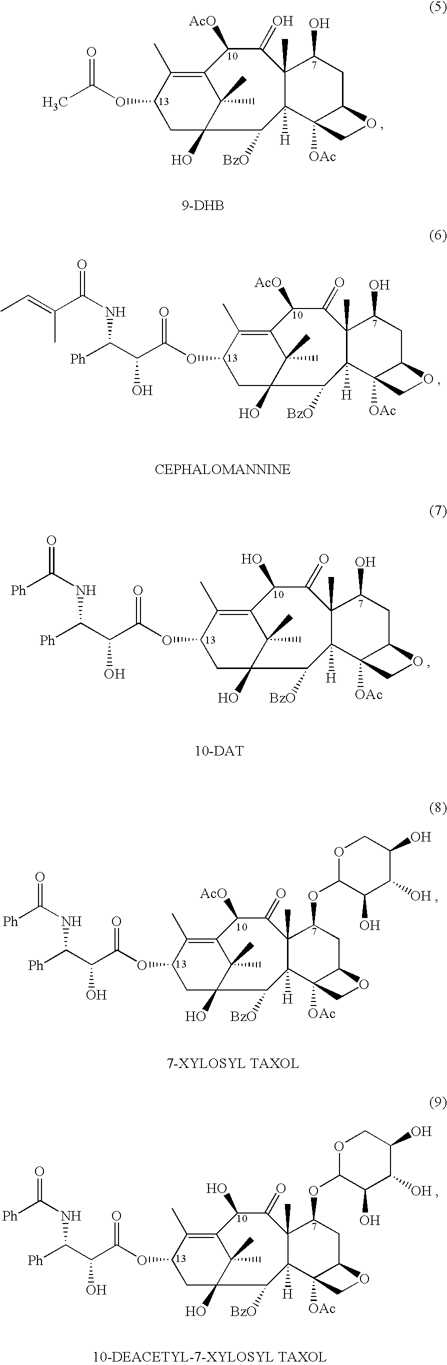

As disclosed in U.S. patent application Ser. No. 10/695,416, which application is assigned to the assignee of the present invention, U.S. Pat. Nos. 6,576,777 and 6,222,053 to Zamir et al. and U.S. Pat. Nos. 6,175,023 and 6,179,981 to Liu et al., docetaxel and paclitaxel (and suitable starting materials for the synthesis thereof) may also be prepared semi-synthetically from 9-dihydro-13-acetylbaccatin III.

In addition, U.S. Pat. Nos. 5,202,448 and 5,256,801 to Carver et al., U.S. Pat. No. 5,449,790 to Zheng et al. and U.S. Pat. No. 6,281,368 to McChesney et al. disclose processes for converting certain taxanes (namely, paclitaxel, cephalomannine, 10-deacetyl taxol and certain 10-deacetyl taxol derivatives) present in partially purified taxane mixtures into 10-deacetylbaccatin III and baccatin III, which may subsequently be utilized in the foregoing semi-synthetic pathways.

Although there have been many advances in the field, there remains a need for new and improved processes for the preparation of taxane intermediates and their conversion to paclitaxel and docetaxel, in particular, for the preparation of such taxane intermediates from crude and partially purified mixtures comprising a plurality of taxanes. The present invention addresses these needs and provides further related advantages.

### BRIEF SUMMARY OF THE INVENTION

In brief, the present invention relates to the semi-synthesis of taxane intermediates useful in the preparation of paclitaxel and docetaxel, in particular, the semi-synthesis of 10-deacetylbaccatin III and baccatin III, and derivatives thereof, from a mixture of taxanes. In this way, the disclosed processes may be utilized to convert a plurality of taxanes present in a crude taxane extract or in a waste taxane solution into taxanes, and taxane derivatives, that can be used to further synthesize paclitaxel and docetaxel. Representative waste taxane solutions may comprise (1) pooled waste stream fractions collected following the chromatographic separation and collection of paclitaxel enriched fractions from a crude or partially purified taxane extract, and/or (2) pooled waste mother liquors collected following the recrystallization of a crude or partially purified taxane extract.

As set forth below, each of the disclosed processes comprise an initial combined step of protecting the hydroxy group at the C-7 position of each taxane in the initial mixture having a hydroxy group at the C-7 position and cleaving the ester linkage at the C-13 position of each taxane in the initial mixture having an ester linkage at the C-13 position.

For example, in a first embodiment, the present invention provides a process for preparing 10-deacetylbaccatin III and baccatin III from an initial mixture of taxanes, wherein the initial mixture comprises 9-dihydro-13-acetylbaccatin III or cephalomannine, and at least one additional taxane selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol, the process comprising the steps of:

(1) protecting the hydroxy group at the C-7 position of each taxane in the initial mixture having a hydroxy group at the C-7 position and cleaving the ester linkage at the C-13 position of each taxane in the initial mixture having an ester linkage at the C-13 position to yield a first intermediate mixture of C-7 protected taxanes;

(2) oxidizing the hydroxy group at the C-9 position of each taxane in the first intermediate mixture having a hydroxy group at the C-9 position to yield a second intermediate mixture of C-7 protected taxanes; and

(3) deprotecting the hydroxy group at the C-7 position of each taxane in the second intermediate mixture to yield 10-deacetylbaccatin III and baccatin III.

In a further embodiment, the step of protecting the hydroxy group at the C-7 position of each taxane in the initial mixture and cleaving the ester linkage at the C-13 position of each taxane in the initial mixture further comprises cleaving the ester linkage at the C-10 position of at least one taxane in the initial mixture having an ester linkage at the C-10 position.

In another further embodiment, cleaving the ester linkage at the C-13 position of each taxane in the initial mixture comprises contacting the initial mixture with a base.

In other further embodiments, the initial mixture comprises: (1) 9-dihydro-13-acetylbaccatin III or cephalomannine, and at least two additional taxanes selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol; (2) 9-dihydro-13-acetylbaccatin III or cephalomannine, and at least three additional taxanes selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol; or (3) 9-dihydro-13-acetylbaccatin III, paclitaxel, 10-deacetylbaccatin III, baccatin III, cephalomannine, 10-deacetyl taxol, 7-xylosyl taxol and 10-deacetyl-7-xylosyl taxol.

In yet another further embodiment, the initial mixture of taxanes is a waste taxane solution comprising one or more of the following: (1) pooled waste stream fractions collected during a chromatographic separation of a crude or partially purified taxane extract; and (2) pooled waste mother liquors collected during a recrystallization of a crude or partially purified taxane extract. In a specific embodiment, the waste taxane solution comprises pooled waste stream fractions collected during a chromatographic separation of a crude taxane extract.

In a more specific embodiment, the step of protecting the hydroxy group at the C-7 position of each taxane in the initial mixture and cleaving the ester linkage at the C-13 position of each taxane in the initial mixture comprises contacting the initial mixture with a base and a hydroxy-protecting group in an organic solvent, wherein the base is selected from the group consisting of DMAP, pyridine, TEA, LiOH, Li-t-OBu, n-BuLi, K-t-OBu and a mixture of n-BuLi/K-t-OBu, and the hydroxy-protecting group is selected from the group consisting of alkylating agents and acylating agents. More specifically, the hydroxy-protecting group is selected from the group consisting of tert-butoxycarbonyl, benzyloxycarbonyl, 2,2,2-trichloroethoxycarbonyl, dichloroacetyl and acetyl. In a specific embodiment, the base is DMAP and the hydroxy-protecting group is tert-butoxycarbonyl.

In another more specific embodiment, the step of oxidizing the hydroxy group at the C-9 position of each taxane in the first intermediate mixture comprises contacting the first intermediate mixture with an oxidizing agent selected from the group consisting of 4-(dimethylamino)pyridinium chlorochromate, pyridinium chlorochromate, chromium (IV) oxide-silica gel, chromium (IV) oxide-acetic acid, bromine, dimethyl sulfoxide-dicyclohexylcarbodiimide, and manganese dioxide with dichloro(p-cymene)-ruthenium (II). In a specific embodiment, the oxidizing agent is chromium (IV) oxide-silica gel.

In yet another more specific embodiment, the step of deprotecting the hydroxy group at the C-7 position of each taxane in the second intermediate mixture comprises contacting the second intermediate mixture with an acid. More specifically, the acid is selected from the group consisting of HF, TFA, HCl and acetic acid.

In a second embodiment, the present invention provides a process for preparing paclitaxel or docetaxel from an initial mixture of taxanes, wherein the initial mixture comprises 9-dihydro-13-acetylbaccatin III or cephalomannine, and at least one additional taxane selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol, the process comprising the steps of:

(1) protecting the hydroxy group at the C-7 position of each taxane in the initial mixture having a hydroxy group at the C-7 position and cleaving the ester linkage at the C-13 position of each taxane in the initial mixture having an ester linkage at the C-13 position to yield a first intermediate mixture of C-7 protected taxanes;

(2) oxidizing the hydroxy group at the C-9 position of each taxane in the first intermediate mixture having a hydroxy group at the C-9 position to yield a second intermediate mixture of C-7 protected taxanes;

(3) deprotecting the hydroxy group at the C-7 position of each taxane in the second intermediate mixture to yield 10-deacetylbaccatin III and baccatin III; and

(4) converting the 10-deacetylbaccatin III and baccatin III to paclitaxel or docetaxel, wherein the step of converting the 10-deacetylbaccatin III and baccatin III to paclitaxel or docetaxel further comprises protecting the hydroxy group at the C-7 position of each of the 10-deacetylbaccatin III and baccatin III.

These and other aspects of the invention will be apparent upon reference to the following detailed description.

### DETAILED DESCRIPTION OF THE INVENTION

I. Definitions

As used herein, the following terms have the following meanings.

“Silica matrix” is a solid media containing a silicate which is used as an adsorbent or column material in chromatographic separations, including (but not limited to) ordinary silica, Florisil, porous silica gels or any physical formulation of a silicate for use in chromatographic procedures.

“Taxane-containing material” refers to selected parts of a plant, plant tissues, cell cultures, microorganisms or extracts with extractable taxanes, including paclitaxel, 10-deacetylbaccatin III (10-DAB), baccatin III (BACC III), 9-dihydro-13-acetylbaccatin III (9-DHB), cephalomannine, 10-deacetyl taxol (10-DAT), 7-xylosyl taxol and 10-deacetyl-7-xylosyl taxol.

“Crude taxane extract” refers to a composition obtained from a taxane-containing material by treating the taxane-containing material with at least one solvent.

“Partially purified taxane extract” refers to a paclitaxel enriched composition obtained from the chromatographic separation and/or recrystallization of a crude or partially purified taxane extract.

“Waste stream fractions” refers to fractions collected following the chromatographic separation and collection of paclitaxel enriched fractions from a crude or partially purified taxane extract by, for example, the process of U.S. Pat. No. 6,136,989.

“Waste mother liquors” refers to mother liquors collected following the recrystallization of a crude or partially purified taxane extract by, for example, the process of U.S. Pat. No. 6,136,989.

“Hydroxy-protecting group” refers to a readily cleavable group bonded to the oxygen of a hydroxy (—OH) group. Examples of hydroxy-protecting groups include, without limitation, formyl, acetyl (Ac), benzyl (PhCH₂), 1-ethoxyethyl (EE), methoxymethyl (MOM), (methoxyethoxy)methyl (MEM), (p-methoxyphenyl)methoxymethyl (MPM), tert-butyldimethylsilyl (TBS), tert-butyldiphenylsilyl (TBPS), tert-butoxycarbonyl (tBoc, t-Boc, tBOC, t-BOC), tetrahydropyranyl (THP), triphenylmethyl (Trityl, Tr), 2-methoxy-2-methylpropyl, benzyloxycarbonyl (Cbz), dichloroacetyl, trichloroacetyl (OCCCl₃), 2,2,2-trichloroethoxycarbonyl (Troc), benzyloxymethyl (BOM), tert-butyl (t-Bu), triethylsilyl (TES), trimethylsilyl (TMS), triisopropylsilyl (TIPS), propionyl, isopropionyl, pivalyl, dimethylisopropylsilyl, diethylisopropylsilyl, methyldiphenylsilyl, dimethylphenylsilyl, tert-butyldiphenylsilyl, tribenzylsilyl, triphenylsilyl, trichloroethoxycarbonyl, benzyl, para-nitrobenzyl, para-methoxybenzyl, benzoyl, methoxyethyl, para-methoxyphenyl, tetrahydrofuranyl, alkylsulfonyl and arylsulfonyl. The related term “protected hydroxy group” refers to a hydroxy group that is bonded to a hydroxy-protecting group. General examples of protected hydroxy groups include, without limitation, —O-alkyl, —O-acyl, acetal, and —O-ethoxyethyl, where some specific protected hydroxy groups include, formyloxy, acetoxy, propionyloxy, chloroacetoxy, bromoacetoxy, dichloroacetoxy, trichloroacetoxy, trifluoroacetoxy, methoxyacetoxy, phenoxyacetoxy, benzoyloxy, benzoylformoxy, p-nitro benzoyloxy, ethoxycarbonyloxy, methoxycarbonyloxy, propoxycarbonyloxy, 2,2,2-trichloro ethoxycarbonyloxy, benzyloxycarbonyloxy, tert-butoxycarbonyloxy, 1-cyclopropyl ethoxycarbonyloxy, phthaloyloxy, butyryloxy, isobutyryloxy, valeryloxy, isovaleryloxy, oxalyoxy, succinyloxy and pivaloyloxy, phenylacetoxy, phenylpropionyloxy, mesyloxy, chlorobenzoyloxy, para-nitrobenzoyloxy, para-tert-butyl benzoyloxy, capryloyloxy, acryloyloxy, methylcarbamoyloxy, phenylcarbamoyloxy, naphthylcarbamoyloxy, and the like. Hydroxy-protecting groups and protected hydroxy groups are described in, e.g., C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” Second Edition, John Wiley and Sons, New York, N.Y., 1991, Chapters 2 and 3.

The following Table shows the chemical structure of some hydroxy-protecting groups, as well as nomenclature used to identify those chemical structures.

TABLE 1

| | |
|---|---|
| Acetyl(Ac) | 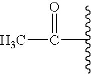 |
| Acetoxy(—OAc) | 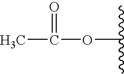 |
| Dichloroacetyl | 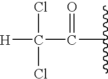 |
| Dichloroacetoxy | 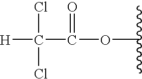 |
| Triethylsilyl(TES) | 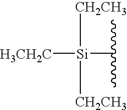 |
| Triethylsiloxy(—OTES) | 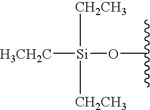 |
| Benzoyl | 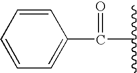 |
| Benzoyloxy | 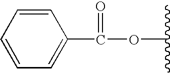 |
| t-Butyloxycarbonyl(tBOC) | 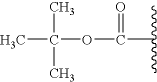 |
| t-Butoxycarbonyloxy(—O—tBOC) | 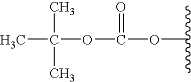 |
| para-Methoxyphenyl(PMP) | 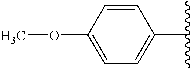 |

“Alkyl” refers to a hydrocarbon structure wherein the carbons are arranged in a linear, branched, or cyclic manner, including combinations thereof. Lower alkyl refers to alkyl groups of from 1 to 5 carbon atoms. Examples of lower alkyl groups include methyl, ethyl, propyl, isopropyl, butyl, s- and t-butyl and the like. “Cycloalkyl” is a subset of alkyl and includes cyclic hydrocarbon groups of from 3 to 13 carbon atoms. Examples of cycloalkyl groups include cyclopropyl, cyclobutyl, cyclopentyl, norbornyl, adamantyl and the like. When an alkyl residue having a specific number of carbons is named, all geometric isomers having that number of carbons are intended to be encompassed; thus, for example, “butyl” is meant to include n-butyl, sec-butyl, isobutyl and t-butyl; propyl includes n-propyl and isopropyl.

“Alkenyl” refers to an alkyl group having at least one site of unsaturation, i.e., at least one double bond.

“Alkynyl” refers to an alkyl group having at least one triple bond between adjacent carbon atoms.

“Alkoxy” and “alkoxyl” both refer to moieties of the formula —O-alkyl. Examples include methoxy, ethoxy, propoxy, isopropoxy, cyclopropyloxy, cyclohexyloxy and the like. Lower-alkoxy refers to groups containing one to four carbons. The analogous term “aryloxy” refers to moieties of the formula —O-aryl.

“Acyl” refers to moieties of the formula —C(═O)-alkyl. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

“Aryl” refers to phenyl or naphthyl. Substituted aryl refers to mono- and poly-substituted phenyl or naphthyl. Exemplary substituents for aryl include one or more of halogen, hydroxyl, alkoxy, aryloxy, heteroaryloxy, amino, alkylamino, dialkylamino, mercapto, alkylthio, arylthio, heteroarylthio, cyano, carboxyl, alkoxycarbonyl where the alkoxy portion contains 1 to 15 carbons, aryloxycarbonyl where the aryloxy portion contains 6 to 20 carbon, or heteroarylcarbonyl where the heteroaryl portion contains 3 to 15 carbon atoms.

“Heteroaryl” refers to a 5- or 6-membered heteroaromatic ring containing 1-3 heteroatoms selected from O, N, or S; a bicyclic 9- or 10-membered heteroaromatic ring system containing 0-3 heteroatoms selected from O, N, or S; or a tricyclic 13- or 14-membered heteroaromatic ring system containing 0-3 heteroatoms selected from O, N, or S. Exemplary aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

“Halogen” refers to fluoro, chloro, bromo or iodo.

II. Process for Preparing 10-DAB and BACC III from a Mixture of Taxanes

As noted above, the present invention relates to the semi-synthesis of taxane intermediates useful in the preparation of paclitaxel and docetaxel, in particular, the semi-synthesis of 10-deacetylbaccatin III and baccatin III, and derivatives thereof, from an initial mixture of taxanes.

The taxanes present in the initial mixture, namely, 9-dihydro-13-acetylbaccatin III, paclitaxel, 10-deacetylbaccatin III, baccatin III, cephalomannine, 10-deacetyl taxol, 7-xylosyl taxol and 10-deacetyl-7-xylosyl taxol, contain the following baccatin molecular framework: 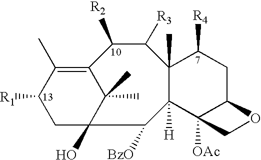
wherein R₁, R₂, R₃ and R₄ represent substituents which vary between the taxanes. More specifically, R₁ is —OH, —OAc, N-(2-methyl-2-butenoyl)-(2R,3S)-3-phenylisoserine or N-benzoyl-(2R,3S)-3-phenylisoserine, R₂ is —OH or —OAc, R₃ is —OH or ═O, and R₄ is —OH or xylosyl. For example, when R₁ is —OAc, R₂ is —OAc, R₃ is —OH and R₄ is —OH, the foregoing structure represents 9-dihydro-13-acetylbaccatin III, and when R₁ is —OH, R₂ is —OAc, R₃ is =0 and R₄ is —OH, the foregoing structure represents baccatin III.

As described in more detail below, 10-deacetylbaccatin III and baccatin III may be prepared from such an initial mixture of taxanes through the protection, cleavage, oxidation and deprotection of certain R₁, R₂, R₃ and R₄ substituents of the taxanes present in the initial mixture. For example, the present invention provides a process for preparing 10-deacetylbaccatin III and baccatin III from an initial mixture of taxanes, wherein the initial mixture comprises 9-dihydro-13-acetylbaccatin III or cephalomannine, and at least one additional taxane selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol, the process comprising the steps of:

(1) protecting the hydroxy group at the C-7 position of each taxane in the initial mixture having a hydroxy group at the C-7 position and cleaving the ester linkage at the C-13 position of each taxane in the initial mixture having an ester linkage at the C-13 position to yield a first intermediate mixture of C-7 protected taxanes;

(2) oxidizing the hydroxy group at the C-9 position of each taxane in the first intermediate mixture having a hydroxy group at the C-9 position to yield a second intermediate mixture of C-7 protected taxanes; and

(3) deprotecting the hydroxy group at the C-7 position of each taxane in the second intermediate mixture to yield 10-deacetylbaccatin III and baccatin III.

In general, such protection, cleavage, oxidation and deprotection steps comprise the following methods.

General Method of Protection and Cleavage

The hydroxy group at the C-7 position of a taxane can be selectively protected using any of a variety of hydroxy protecting groups, such as acetal, ketal, silyl, and removable acyl protecting groups. For example, the C-7 hydroxy group may be silylated using any of a variety of common silylating agents including, but not limited to, tri(hydrocarbonyl)silyl halides and tri(hydrocarbonyl)silyl triflates. The hydrocarbonyl moieties of these compounds may be substituted or unsubstituted and preferably are substituted or unsubstituted alkyl or acyl. More specifically, the C-7 hydroxy group can be selectively silylated, for example, using silylating agents such as tribenzylsilyl chloride, trimethylsilyl chloride, triethylsilyl chloride, dimethylisopropylsilyl chloride, dimethylphenylsilyl chloride and the like. Alternatively, selective acylation of the C-7 hydroxy group can be achieved using any of a variety of common acylating agents, but not limited to substituted and unsubstituted carboxylic acid derivatives, e.g., carboxylic acid halides, anhydrides, dicarbonates, isocyanates and haloformates. More specifically, the C-7 hydroxy group can be selectively acylated, for example, with di-tert-butyl dicarbonate, dibenzyl dicarbonate, diallyl dicarbonate, 2,2,2-trichloroethyl chloroformate, benzyl chloroformate or dichloroacetyl chloride or another common acylating agent.

In the present invention, these protecting reactions are carried out in the presence of a base, such as, for example, Li-t-OBu, K-t-OBu, n-BuLi, a mixture of n-BuLi/K-t-OBu, LiOH, pyridine, DMAP or TEA. In addition to aiding in the protection of the C-7 hydroxy group, depending upon the amount of base utilized, the base also cleaves any ester linkage at the C-13 position and, may also cleave any ester linkage at the C-10 position.

Exemplary reaction conditions are as follows: a mixture of taxanes is dissolved in anhydrous DCM (dichloromethane) or THF (tetrahydrofuran) or DMF (dimethyl formamide) or DMSO (dimethyl sulfoxide) under an argon atmosphere at low temperature. To this solution is added DMAP (dimethylaminopyridine) or any other lithium or potassium base, such as Li-t-OBu, K-t-OBu, n-BuLi, a mixture of n-BuLi/K-t-OBu, or LiOH, followed by an acylating agent, such as di-tert-butyl dicarbonate, or an alkylating agent, such as triethyl silyl chloride or any other chloride containing a hydroxy-protecting group. The mixture is left at low to room temperature until complete consumption of the starting material, as visualized by TLC. In addition, excess amounts of the base may be added to this mixture, in the same pot, to ensure cleavage of any ester linkages at the C-13 and/or C-10 positions. The mixture is then quenched with cold water and extracted thrice with DCM. The organic layer is washed with water and then with brine to remove unwanted salts. The organic layer may then be dried and evaporated under vacuum, and the residue recrystallized or column chromatographed with DCM/EtOAc mixtures to afford a mixture of C-7 protected taxanes.

General Method of Oxidation

C-7 protected taxanes may be selectively oxidized at the C-9 position using various oxidizing agents, such as 4-(dimethylamino)pyridinium chlorochromate, pyridinium chlorochromate, chromium (IV) oxide-silica gel, chromium (IV) oxide-acetic acid (Fieser reagent) or acidic media, bromine, dimethyl sulfoxide-dicyclohexylcarbodiimide, and manganese dioxide with dichloro(p-cymene)-ruthenium (II).

For example, C-7 protected taxanes are dissolved in an organic solvent and treated with an oxidant at low to room temperature conditions. The reaction is stirred until all the starting material is consumed, as evidenced by TLC. The reaction is then worked up as usual to yield an oxidized mixture of C-7 protected taxanes. Such mixture can be further purified by column chromatography or crystallized from a suitable solvent.

General Method of Deprotection

C-7 protected taxanes may be deprotected using an acid. Suitable acids include HF, HCl, TFA and acetic acid.

For example, C-7 protected taxanes are dissolved in pyridine or an organic solvent at room temperature and treated with an acid, such as HF, HCl, TFA or acetic acid. The reaction is stirred at this temperature until complete consumption of the starting materials, as evidenced by TLC. The reaction is worked up as usual to give the deprotected product, which could be further purified by column chromatography or crystallized from a suitable solvent.

III. Initial Mixture of Taxanes

As noted above, the processes of the present invention may be utilized for high yield and large scale conversion of taxanes present in a waste taxane solution into taxanes, namely, 10-deacetylbaccatin III and baccatin III, that can be used to further synthesize paclitaxel and docetaxel. Such a waste taxane solution may comprise (1) pooled waste stream fractions collected following the chromatographic separation and collection of paclitaxel enriched fractions from a crude or partially purified taxane extract, and/or (2) pooled waste mother liquors collected following the recrystallization of a crude or partially purified taxane extract.

Such waste taxane solutions may be obtained by a number of different methods, such as, for example, the methods disclosed in U.S. Pat. No. 6,136,989 to Foo et al., and other references cited therein, which patent is incorporated herein by reference in its entirety. A representative method of obtaining a waste taxane solution, which comprises pooled waste stream fractions, comprises the following extraction and column chromatography steps.

Starting Taxane-Containing Material

A suitable taxane-containing material is any tissue that contains a high taxane content. Examples of suitable taxane-containing material include tissues from various species of Yew plants comprising the genus Taxus, most preferably the roots and needles of ornamental Yew plants such as T. canadensis, T. x media spp Hicksii, T. x dark green spreader and Hill., T. chinensis, T. wallichiana, T. cuspidata, T. globosa, T. sumatrana, T. marei and T. floridana, and the bark of T. brevifolia or T. yunnanensis. Other suitable material include cultures of plant tissues obtained from a Taxus species.

In a typical practice, such as set forth in U.S. Pat. No. 6,139,989, the taxane-containing material is either pulverized, chipped or otherwise ground into small pieces so as to increase efficiency of a solvent extraction. The taxane-containing material may also optionally be dried. Taxane-containing cell culture, cells, microorganisms and fermentation broths will typically be concentrated prior to solvent extraction. Cells and microorganisms can be processed as whole cells or cell paste or pulver.

Extraction

The taxane-containing material may be initially extracted by contacting the material with an organic solvent, usually for a prolonged period of at least 8 hours and typically for about 3 days with or without physical agitation to promote formation of a crude organic extract containing a plurality of taxanes. The extraction may employ any of the solvent systems that are known to be used for the extraction of paclitaxel, including but not limited to, acetone, methanol, ethanol, ethyl acetate, methylene chloride, chloroform, mixtures thereof, and mixtures containing an aqueous component of up to 60%. These solvents are typically added in an amount of about 4-20 liter per kg of the taxane-containing material to prepare the crude organic extract. Reference is made for example, to U.S. Pat. No. 6,136,989 and the publications cited therein which provide a non-exclusive description of several solvent systems that may be used to prepare an organic extract containing a plurality of taxanes.

In one embodiment, the organic solvent is a polar organic solvent, typically an alcohol. For some embodiments, methanol is preferred because of its low cost, ease of removal and efficiency of taxane extraction. In one embodiment, about 6-15 liters of methanol is added for every kg of taxane-containing material to be extracted. The extraction is accelerated by agitating the taxane-containing material, for example, by stirring or percolating the methanol with the taxane-containing material for about 1-5 days at a temperature between room temperature and about 60° C., most typically at about 40° C. When the taxane-containing material contains a paclitaxel content of at least 0.005%, methanol extraction for three days as described above recovers at least 90% of the available paclitaxel from the taxane-containing material, in addition to a plurality of other taxanes, to form a crude methanol extract containing about 0.1-0.5% paclitaxel and having an overall solid content of about 0.5-5% (w/v).

The large volume of methanol extract thus obtained is optionally concentrated, typically about 10-30 fold by evaporation to obtain a methanol extract concentrate having a solid content of about 100-400 g/L.

Liquid-Liquid Extraction

The crude organic extract may be subsequently enriched for taxanes by performing 1-3 liquid-liquid extractions by mixing the organic extract with a non-miscible, organic solvent to form a two phase system wherein one phase contains the plurality of taxanes. Generally, the two phase system includes a polar phase. Optionally, the taxane-containing phase is selected and concentrated by evaporation to form a concentrated extract having a solid content of about 100-400 g/L and a paclitaxel purity of about 1-4%. In some embodiments, water is included to help remove preferentially water soluble materials and the less polar solvent is selected to remove undesirable compounds such as waxes, lipids, pigments, and sterols that are found in different amounts depending on the taxane-containing material used. Typical solvents for liquid-liquid partitioning include hexane, hexanes, and methylene chloride. Methylene chloride has generally been found to be suitable for liquid-liquid extraction of taxane-containing material especially when the solvent used for the crude organic extract is an alcohol.

The concentrated extract obtained is optionally evaporated and the residue is re-dissolved in a solvent for loading onto a silica chromatography matrix.

Other example methods of performing a liquid-liquid extraction are illustrated in U.S. Pat. Nos. 5,475,120, 5,380,916, and 5,670,673 to Rao and references cited therein, and also in U.S. Pat. Nos. 5,618,538 and 5,480,639 to ElSohly et al. and references cited therein. These methods or variants thereof may alternatively be used in lieu of the embodiments described. Furthermore, liquid-liquid extraction may be omitted altogether when a plant extract containing high taxane levels is obtained by other methods such as for example, by intervening precipitation, crystallization or chromatography steps. One example of such a method is found in PCT Publication Nos. WO 98/07712 by Zamir et al, which uses a precipitation step immediately after obtaining an initial organic extract to obtain a paclitaxel fraction that may be about 1% or higher.

Silica Gel Column Chromatography

As further set forth in U.S. Pat. No. 6,136,989, the concentrated extract may be further purified by normal phase silica chromatography. As used herein, silica chromatography generally refers to the process of contacting a sample dissolved in a feed solvent with a silica matrix then eluting the silica matrix with an eluting solvent to obtain a fraction enriched with a desired component.

The dimensions of the first silica column are selected according to the quantity and purity of the solids to be separated. In one embodiment of a pilot scale process, about 250 grams of solids are dissolved in about 0.75 liters of feed solvent which is then chromatographed over a Silica column of about 1.5-inches x 10-feet. In another embodiment, about 40-50 kg of solids are dissolved in about 100-200 liters of feed solvent, and chromatographed over a Silica column of about 18-inches x 10-feet.

It has also been shown that a layer of about 1-15 cm of Celite, preferably about 2-8 cm, on top of the silica column is recommended as a column prefilter which substantially decreases the loading time of the sample. It has further been shown that the optimal eluting solvent for the Silica column should be a hexane/acetone mixture at a ratio of about 3:1 or a DCM/ethyl acetate mixture at a ratio of about 7:3. The “heart cut” fractions containing at least 2% paclitaxel are pooled and further purified, for example, according to the process set forth in U.S. Pat. No. 6,136,989. The remaining waste stream fractions, which contain a plurality of taxanes, including, paclitaxel, 10-deacetylbaccatin III (10-DAB), baccatin III (BACC III), 9-dihydro-13-acetylbaccatin III (9-DHB), cephalomannine, 10-deacetyl taxol (10-DAT), 7-xylosyl taxol and 10-deacetyl-7-xylosyl taxol are pooled into a waste taxane solution for further processing according to the present invention.

Further Purification Steps

As set forth in more detail in U.S. Pat. No. 6,139,989, the paclitaxel enriched “heart cut” fractions obtained from the foregoing chromatography step may be further purified through one or more additional chromatographic or recrystallization steps. Any waste stream fractions or waste mother liquors collected during such additional purification steps may also be pooled and added to the waste taxane solution for further processing according to the present invention.

IV. Process for Preparing Paclitaxel or Docetaxel

As noted above, the mixture of 10-deacetylbaccatin III and baccatin III prepared from an initial mixture of taxanes according to the foregoing process may be utilized to further synthesize paclitaxel and docetaxel. In this way, the present invention provides an overall process for preparing paclitaxel or docetaxel from an initial mixture of taxanes, wherein the initial mixture comprises 9-dihydro-13-acetylbaccatin III or cephalomannine, and at least one additional taxane selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol, the process comprising the steps of:

(1) protecting the hydroxy group at the C-7 position of each taxane in the initial mixture having a hydroxy group at the C-7 position and cleaving the ester linkage at the C-13 position of each taxane in the initial mixture having an ester linkage at the C-13 position to yield a first intermediate mixture of C-7 protected taxanes;

(2) oxidizing the hydroxy group at the C-9 position of each taxane in the first intermediate mixture having a hydroxy group at the C-9 position to yield a second intermediate mixture of C-7 protected taxanes;

(3) deprotecting the hydroxy group at the C-7 position of each taxane in the second intermediate mixture to yield 10-deacetylbaccatin III and baccatin III; and

(4) converting the 10-deacetylbaccatin III and baccatin III to paclitaxel or docetaxel,

wherein the step of converting the 10-deacetylbaccatin III and baccatin III to paclitaxel or docetaxel further comprises protecting the hydroxy group at the C-7 position of each of the 10-deacetylbaccatin III and baccatin III.

10-deacetylbaccatin III and baccatin III may be converted to paclitaxel and docetaxel by a number of different methods, such as, for example, the methods disclosed in U.S. Pat. Nos. 4,924,011, 4,924,012, 5,175,315 and 5,466,834, which patents are incorporated herein by reference in their entirety, and U.S. patent application Ser. No. 10/683,865, which application is assigned to the assignee of the present invention and is incorporated herein by reference in its entirety.

### EXAMPLES

The following Examples disclose specific processes for synthesizing 10-deacetylbaccatin III and baccatin III, and derivatives thereof, from a solution containing a plurality of taxanes, and their subsequent conversion to paclitaxel and docetaxel. Unless otherwise noted, all scientific and technical terms have the meanings as understood by one of ordinary skill in the art.

### Example 1

Extraction of Taxanes from Taxus Canadensis

Needles from the Canadian yew (Taxus Canadensis) were collected in Quebec. The dried needles (3 kg) were extracted by percolation with methanol at room temperature three times using 10 L, 6 L and 6 L volumes of methanol in a glass container equipped with a filter at the bottom with a tap. The extraction with each subsequent volume of methanol was left for 24 hours and the mixture was filtered into an erlenmeyer flask by opening the tap at the bottom to give a crude extract. The crude methanolic extracts were combined and concentrated to give about 1.1 L of a crude methanol extract concentrate.

### Example 2A

Filtration of the Crude Extract

A silica/charcoal filter was prepared as follows. Norit SA3 charcoal (0.5 kg: 100 mesh—Aldrich) was mixed with celite (0.5 kg: AC 2098T—Anachemia) and placed into a coarse scintered glass funnel. The charcoal-celite mixture was soaked with dichloromethane:methanol (9:1) and washed with an additional 1.0 L of the same solvent. The crude methanol extract concentrate was filtered on this bed of charcoal and then washed with 1.5 L of dichloromethane:methanol (9:1). The collected mixture was evaporated under vacuum using a rotovap and the residue was left under high vacuum for one hour using a vacuum pump to remove all traces of methanol.

### Example 2B

Liquid-Liquid Extraction

The crude methanolic extract concentrate was partitioned with a mixture containing methanol (400 ml), water (800 ml) and hexane (1100 ml) in a 5 L separatory funnel. After allowing for the solvents to partition, the top layer with dark green color was tested and discarded, the lower aqueous phase was extracted with methylene chloride two times. The methylene chloride extracts from two partitions were combined and then concentrated to generate 270 ml of DCM extract concentrate containing the plurality of taxanes.

### Example 3

Silica Gel Column Chromatography

318 g of silica gel (40-63 μm) was used to pack a lab 2-feet long column and 70 ml of the DCM extract concentrate (˜21 g solid) was loaded onto the column followed by DCM/EtOAc elution: 7 L of DCM/EtOAc 7:3 and 3 L of DCM/EtOAc 1:1. 100 fractions were collected, each having a 100 ml volume. According to HPLC and TLC analyses, fractions were combined into five groups: paclitaxel containing fractions, cephalomannine and paclitaxel containing fractions, 9-DHB containing fractions, baccatin III containing fractions and 10-DAB containing fractions. The last four fractions (i.e., the waste stream fractions) can be combined into a pooled waste stream solution containing a plurality of taxanes, or can be used individually in further synthetic conversions. In the present case, after the paclitaxel containing fractions were eluted from the column, the remaining fractions were collected and pooled to form a waste taxane solution, which was further utilized in the following steps.

### Example 4

### Synthesis of 10-Deacetylbaccatin III and Baccatin III from Waste Taxane Solution

Protection of C-7 Hydroxy Groups and Cleavage of C-13 Ester Linkages

The waste taxane solution obtained from the above process was dissolved in THF and stirred at −40° C. under argon atmosphere. To this stirred solution at −40° C. was added a base (such as DMAP, pyridine, TEA or any other lithium or potassium base, such as LiOH, Li-t-OBu, n-BuLi, K-t-OBu or a mixture of n-BuLi/K-t-OBu) followed by addition of a hydroxy-protecting group agent (such as triethylsilyl chloride or any other alkylating agent, or acetic anhydride, acetyl chloride, di-tert-butyl dicarbonate or any other acylating agent). The reaction was stirred at this temperature for a period between 30 minutes to 6 hrs until complete consumption of the initial starting material as evidenced by TLC and HPLC analysis. Additional amounts of the base and alkylating or acylating agent were added at this temperature, as necessary for complete consumption of the starting materials and to ensure cleavage of the C-13 ester linkages. Further amounts of the base may be added to ensure cleavage of the C-10 ester linkages. The reaction mixture was then worked up as usual and the solvent removed to give a crude first intermediate mixture of C-7 protected taxanes that could be used directly in the next step of the synthesis or purified by either column chromatography using mixtures of DCM/EtOAc or crystallized from a suitable solvent.

Oxidation of C-9 Hydroxy Groups

The first intermediate mixture of C-7 protected taxanes was oxidized using an oxidizing agent to obtain a ketone at the C-9 position. This oxidation for the first intermediate mixture of C-7 protected taxanes can be achieved using a variety of oxidants under mild conditions, including 4-(dimethylamino)pyridinium chlorochromate, pyridinium chlorochromate, chromium (IV) oxide-silica gel, chromium (IV) oxide-acetic acid (Fieser reagent) or acidic media, bromine, dimethyl sulfoxide-dicyclohexylcarbodiimide, and manganese dioxide with dichloro(p-cymene)-ruthenium (II).

The first intermediate mixture of C-7 protected taxanes was dissolved in an organic solvent (such as acetone) and cooled to near 0° C. with continuous stirring. The oxidant was added to this solution at low temperature and the reaction was stirred for a period between 30 minutes to 6 hrs until complete consumption of the starting materials as evidenced by TLC. After completion of the reaction, the reaction was worked up as usual to afford a crude second intermediate mixture of C-7 protected taxanes that could be further purified (using silica gel column chromatography with mixtures of DCM/EtOAc for elution or crystallization from a suitable solvent) or used directly for the next step of the synthesis.

Deprotection of C-7 Hydroxy Groups

As noted above, deprotection of the C-7 hydroxy groups in the second intermediate mixture of C-7 protected taxanes can be accomplished by using an acid, such as, HF, HCl, TFA or acetic acid.

The second intermediate mixture of C-7 protected taxanes was dissolved in pyridine or an organic solvent at room temperature. To this solution was added HF, or any other acid such as HCl, TFA or acetic acid. After complete consumption of the starting material, as evidenced by TLC, the reaction was worked up as usual and purified by flash chromatography using mixtures of DCM/EtOAc to afford a mixture of 10-deacetylbaccatin III and baccatin III, that could further be crystallized from a suitable solvent to give a product of >99% purity.

### Example 5

### Synthesis of Paclitaxel and Docetaxel from 10-Deacetylbaccatin III and Baccatin III

The mixture of 10-deacetylbaccatin III and baccatin III is then converted to paclitaxel and/or docetaxel according to, for example, the process set forth in U.S. Pat. No. 5,466,834, which application is incorporated herein by reference in its entirety.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A process for preparing 10-deacetylbaccatin III and baccatin III from an initial mixture of taxanes, wherein the initial mixture comprises 9-dihydro-13-acetylbaccatin III or cephalomannine, and at least one additional taxane selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol, the process comprising the steps of:
protecting the hydroxy group at the C-7 position of each taxane in the initial mixture having a hydroxy group at the C-7 position and cleaving the ester linkage at the C-13 position of each taxane in the initial mixture having an ester linkage at the C-13 position to yield a first intermediate mixture of C-7 protected taxanes;
oxidizing the hydroxy group at the C-9 position of each taxane in the first intermediate mixture having a hydroxy group at the C-9 position to yield a second intermediate mixture of C-7 protected taxanes; and
deprotecting the hydroxy group at the C-7 position of each taxane in the second intermediate mixture to yield 10-deacetylbaccatin III and baccatin III.

2. The process of claim 1 wherein the step of protecting the hydroxy group at the C-7 position of each taxane in the initial mixture and cleaving the ester linkage at the C-13 position of each taxane in the initial mixture further comprises cleaving the ester linkage at the C-10 position of at least one taxane in the initial mixture having an ester linkage at the C-10 position.

3. The process of claim 1 wherein:
the step of protecting the hydroxy group at the C-7 position of each taxane in the initial mixture and cleaving the ester linkage at the C-13 position of each taxane in the initial mixture comprises contacting the initial mixture with a base and a hydroxy-protecting group in an organic solvent;
the base is selected from the group consisting of DMAP, pyridine, TEA, LiOH, Li-t-OBu, n-BuLi, K-t-OBu and a mixture of n-BuLi/K-t-OBu; and
the hydroxy-protecting group is selected from the group consisting of alkylating agents and acylating agents.

4. The process of claim 3 wherein the hydroxy-protecting group is selected from the group consisting of tert-butoxycarbonyl, benzyloxycarbonyl, 2,2,2-trichloroethoxycarbonyl, dichloroacetyl and acetyl.

5. The process of claim 4 wherein the base is DMAP and the hydroxy-protecting group is tert-butoxycarbonyl.

6. The process of claim 1 wherein cleaving the ester linkage at the C-13 position of each taxane in the initial mixture comprises contacting the initial mixture with a base.

7. The process of claim 1 wherein the step of oxidizing the hydroxy group at the C-9 position of each taxane in the first intermediate mixture comprises contacting the first intermediate mixture with an oxidizing agent selected from the group consisting of 4-(dimethylamino)pyridinium chlorochromate, pyridinium chlorochromate, chromium (IV) oxide-silica gel, chromium (IV) oxide-acetic acid, bromine, dimethyl sulfoxide-dicyclohexylcarbodiimide, and manganese dioxide with dichloro(p-cymene)-ruthenium (II).

8. The process of claim 7 wherein the oxidizing agent is chromium (IV) oxide-silica gel.

9. The process of claim 1 wherein the step of deprotecting the hydroxy group at the C-7 position of each taxane in the second intermediate mixture comprises contacting the second intermediate mixture with an acid.

10. The process of claim 9 wherein the acid is selected from the group consisting of HF, TFA, HCl and acetic acid.

11. The process of claim 1 wherein the initial mixture comprises 9-dihydro-13-acetylbaccatin III or cephalomannine, and at least two additional taxanes selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol.

12. The process of claim 1 wherein the initial mixture comprises 9-dihydro-13-acetylbaccatin III or cephalomannine and at least three additional taxanes selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol.

13. The process of claim 1 wherein the initial mixture comprises 9-dihydro-13-acetylbaccatin III, paclitaxel, 10-deacetylbaccatin III, baccatin III, cephalomannine, 10-deacetyl taxol, 7-xylosyl taxol and 10-deacetyl-7-xylosyl taxol.

14. The process of claim 1 wherein the initial mixture of taxanes is a waste taxane solution comprising one or more of the following:
pooled waste stream fractions collected during a chromatographic separation of a crude or partially purified taxane extract; and
pooled waste mother liquors collected during a recrystallization of a crude or partially purified taxane extract.

15. The process of claim 14 wherein the waste taxane solution comprises pooled waste stream fractions collected during a chromatographic separation of a crude taxane extract.

16. The process of claim 14 wherein the waste taxane solution comprises pooled waste stream fractions collected during chromatographic separations of both crude and partially purified taxane extracts and pooled waste mother liquors collected during recrystallizations of both crude and partially purified taxane extracts.

17. The process of claim 14 wherein the crude and partially purified taxane extracts are obtained from taxane-containing materials from the genus Taxus.

18. A process for preparing paclitaxel or docetaxel from an initial mixture of taxanes, wherein the initial mixture comprises 9-dihydro-13-acetylbaccatin III or cephalomannine, and at least one additional taxane selected from paclitaxel, 10-deacetylbaccatin III, baccatin III, 9-dihydro-13-acetylbaccatin III, cephalomannine, 10-deacetyl taxol, 7-xylosyl taxol and 10-deacetyl-7-xylosyl taxol, the process comprising the steps of:
protecting the hydroxy group at the C-7 position of each taxane in the initial mixture having a hydroxy group at the C-7 position and cleaving the ester linkage at the C-13 position of each taxane in the initial mixture having an ester linkage at the C-13 position to yield a first intermediate mixture of C-7 protected taxanes;
oxidizing the hydroxy group at the C-9 position of each taxane in the first intermediate mixture having a hydroxy group at the C-9 position to yield a second intermediate mixture of C-7 protected taxanes;
deprotecting the hydroxy group at the C-7 position of each taxane in the second intermediate mixture to yield 10-deacetylbaccatin III and baccatin III; and
converting the 10-deacetylbaccatin III and baccatin III to paclitaxel or docetaxel,
wherein the step of converting the 10-deacetylbaccatin III and baccatin III to paclitaxel or docetaxel further comprises protecting the hydroxy group at the C-7 position of each of the 10-deacetylbaccatin III and baccatin III.
